(19) United States
(12) Patent Application Publication
(10) Pub. No.: US 2012/0323242 A1
(43) Pub. Date: Dec. 20, 2012

(54) SURGICAL AWL AND METHOD OF USING THE SAME

(75) Inventors: Yang-Hwei Tsuang, Taipei City (TW); Chun-Jen Liao, Taipei City (TW); Huang-Chien Liang, Hsinchu City (TW); Shih-Jui Han, Taichung City (TW); Fon-Yih Tsuang, Taipei City (TW); Chang-Jung Chiang, Changhua County (TW)

(73) Assignee: Industrial Technology Research Institute, Hsinchu (TW)

(21) Appl. No.: 13/161,698

(22) Filed: Jun. 16, 2011

(51) Int. Cl. A61B 17/16

(52) U.S. Cl. 606/79

(57) ABSTRACT

A bone awl is provided for preparing a bone for implantation with a screw. The awl includes a shaft having a first end and a second end opposed to the first end. A handle is fixed to the first end, and an extension extends from the second end. The extension includes a first cutting portion configured to form a hole in bone and a second cutting portion configured to form an internal screw thread in the bone along the surface of the hole. The awl also includes an axial through passage that extends from the handle to the first cutting portion and is dimensioned to receive a Kirshner pin therein.

### BACKGROUND

Each year 13 million people see a doctor for chronic back pain, which is estimated to cause 2.4 million Americans to be chronically disabled. About 25 percent of people who have back pain have a herniated disk. In the US, about 450 cases of herniated disk per 100,000 require surgery such as a discectomy.

Referring to FIG. 1, a discectomy (FIG. 1(a)) is performed when the intervertebral disc 8 has herniated or torn and has not responded to a more conservative treatment. When a surgeon performs a discectomy, it is usually performed through an incision in the patient's back at a location corresponding to the problem area of the spine 2. Muscles and ligaments are moved aside to expose the offending disc 8. The surgeon then uses a variety of surgical instruments to first separate the vertebrae 4 sandwiching the disc 8, and then remove the disc 8 completely. After a discectomy is performed, the spinal column at the operation site is separated to approximate the height of the removed disc (FIG. 1b), and then an artificial disk may be placed in the separation. Spinal fixation devices (FIG. 1(c)) are used to stabilize and/or align the spine 2 during the healing process following such procedures. In some cases, clinicians fill the separation with the implantation of autologous bone to achieve fusion (fusion is illustrated in FIG. 1(d)) to restore stability of the spine 2. Alternatively, discectomy may be may be followed by spinal fusion, or other procedure that may be deemed necessary to strengthen and straighten the spinal canal.

Although a discectomy is frequently performed using minimally invasive devices and procedures, it is still challenging to provide the minimally invasive spine stabilization that is required following this and other spinal procedures. Improved tools for use during implantation of minimally invasive spinal fixation devices are required to minimizing patient risk, trauma, recovery time, and to reduce the overall costs of such procedures.

### SUMMARY

In some aspects, a bone awl is provided that includes a shaft having a first end and a second end opposed to the first end. A handle is fixed to the second end, and an extension extends from the first end that includes a first cutting portion configured to form a hole in bone and a second cutting portion configured to form an internal screw thread in the bone along the surface of the hole.

The bone awl may include one or more of the following features: The bone awl may further comprise an axial through passage that extends from the handle to the first cutting portion. The first cutting portion defines a terminus of the awl, and the second cutting portion is disposed between the first cutting portion and the shaft first end. The first and second cutting portions are selectively detachable from the shaft. The second cutting portion includes tap threads. An external surface of the handle includes surface features configured to improve gripability. The handle includes a detachable cap. The detachable cap is configured to releasably engage an end of a Kirshner pin. The handle is hollow and includes a distal end that is fixed to the shaft second end, a proximal end that is opposed to the distal end, the proximal end being open to provide access to the interior space of the handle, and a detachable cap that closes the open proximal end. The interior space is threaded, and the cap includes threads configured to engage the threads of the interior space. The cap is configured to releasably engage an end of a Kirshner pin. The bone awl further includes a Kirshner pin disposed within the axial through passage.

In some aspects, a method of implanting a pedicle screw into a vertebra using a surgical awl is provided. The method includes the following steps: Providing the pedicle screw. Providing the surgical awl, the surgical awl including a hollow body including a proximal end, and a distal end opposed to the proximal end, the proximal end including a handle, and the distal end including a first cutting portion configured to form a hole in bone and a second cutting portion configured to form an internal screw thread in the bone along the surface of the hole, a detachable cap formed on the handle, and a first Kirshner pin extending through the hollow body to the distal end. Forming an incision through the skin overlying the vertebra. Inserting the awl into the incision until the distal end contacts the vertebra. Rotating the awl so that the first cutting portion forms a hole in the vertebra, and the second cutting portion forms an internal screw thread in the hole formed by the first cutting portion. Removing the cap from a proximal end of the awl. Removing the first Kirshner pin from the awl. Inserting a second Kirshner pin through the awl and into the vertebra at the desired implantation location. Removing the awl from the incision, leaving the second Kirshner pin in place. Implanting the pedicle screw in the pre-threaded drill hole of the vertebra by passing it along the second Kirshner pin and screwing the pedicle screw into the hole in the vertebra.

The method of implanting a pedicle screw into a vertebra may include one or more additional features and/or method steps: The cap is configured to detachably retain an end of the first Kirshner pin, and when the cap is removed from the proximal end of the awl, the first Kirshner pin is removed along with the cap. The method further includes a tissue dilation step following the removal of the awl from the incision. The tissue dilation step includes inserting a series of dilation cannulas into the incision over the second Kirshner pin, starting with a relatively small-diameter cannula, and each successive cannula having a slightly larger outer diameter, and after dilation is completed, withdrawing the dilation cannulas and leaving the second Kirshner pin in place. The method further includes verifying correct awl positioning using an imaging device following the step of inserting the awl into the incision.

In some aspects, a method of using a surgical awl to form a tapped hole in bone is provided. The method includes the following method steps: Providing the surgical awl, the surgical awl including a proximal end, and a distal end opposed to the proximal end, the distal end including a first cutting portion configured to form a hole in bone and a second cutting portion configured to form an internal screw thread in the bone along the surface of the hole. Placing the distal end against a surface of the bone. Rotating the awl so that the first cutting portion forms a hole in the bone. After forming the hole in the bone, further rotating the awl so that the second cutting portion forms an internal screw thread in the hole formed by the first cutting portion.

An awl is disclosed that facilitates implantation of a screw in a bone. In the illustrated embodiment, the awl is used to facilitate implantation of a pedicle screw in a vertebra, as is required for implantation of a spinal fixation system. The awl advantageously incorporates both a drilling portion and a tap thread portion on the same shaft, which includes elongated portions that facilitate stabilizing the direction of implantation. In addition, the awl can receive a Kirshner pin to further facilitate achieving accurate implant location and orientation.

Modes for carrying out the present invention are explained below by reference to an embodiment of the present invention shown in the attached drawings. The above-mentioned object, other objects, characteristics and advantages of the present invention will become apparent from the detailed description of the embodiment of the invention presented below in conjunction with the attached drawings.

### BRIEF DESCRIPTION OF THE DRAWINGS

FIGS. 1 (a)-1(d) illustrate procedures for repairing a herniated disk.

FIG. 2 is a perspective view of a minimally invasive spinal fixation system implanted on a series of four adjacent vertebrae.

FIG. 3 is a side view of a pedicle rod used in the spinal fixation system of FIG. 2.

FIG. 4 is an exploded perspective view of a pedicle screw assembly including a pedicle screw as used in the spinal fixation system of FIG. 2, a cap and a stabilizer tool.

FIG. 4a is a cross-sectional view of the pedicle screw assembly of FIG. 4, as seen across line 4a-4a.

FIG. 5 is a perspective view of a fastener used in the spinal fixation system of FIG. 2.

FIG. 6a is an exploded view of a breaking tool used during implantation of the spinal fixation system of FIG. 2.

FIG. 6b is an enlarged view of the portion of the breaking tool marked as 6b in FIG. 6a.

FIG. 6c illustrates the breaking tool in use separating the pedicle head into two portions.

FIG. 7 is a side view of an awl used during implantation of the spinal fixation system of FIG. 2.

FIG. 8 is a detail view of the tip of the awl of FIG. 7.

FIG. 9 is a detail view of the handle of the awl of FIG. 7 with the cap detached.

FIGS. 10-22 illustrate method steps of using the awl to implant a pedicle screw in a vertebra.

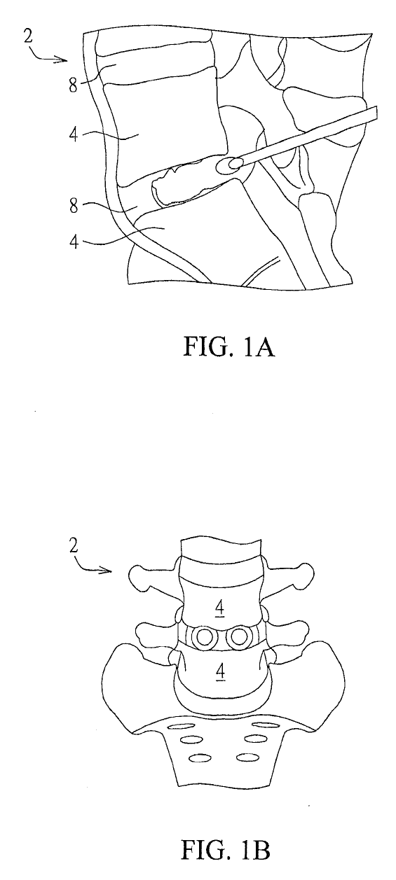

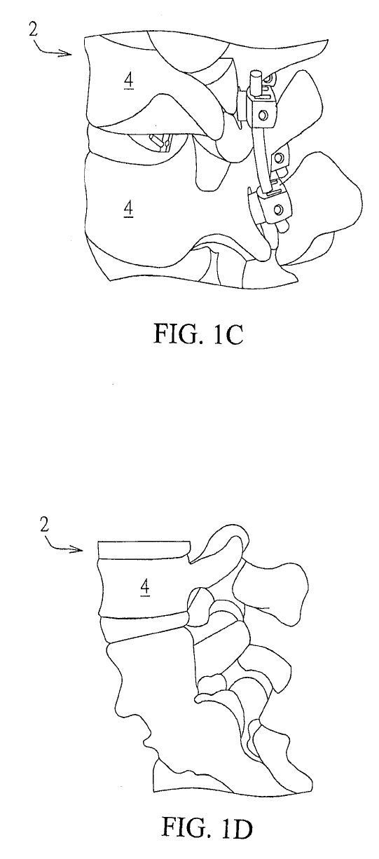

### DETAILED DESCRIPTION

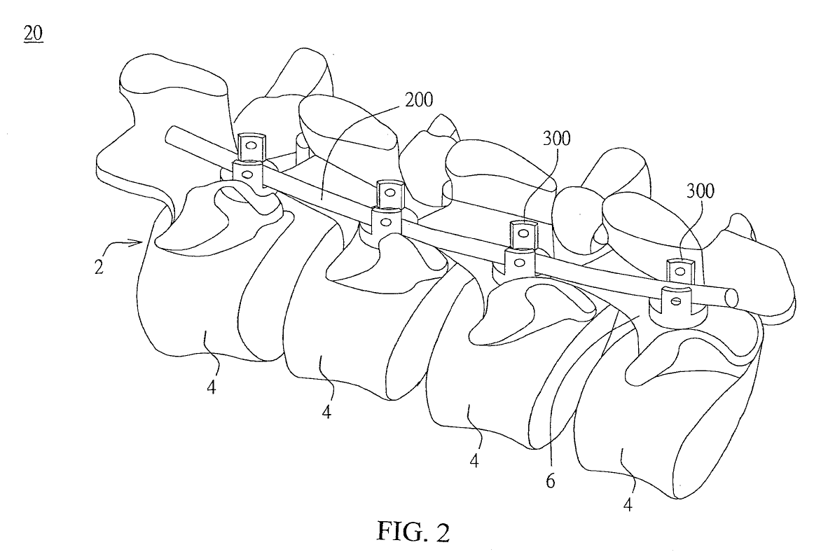

Referring to FIG. 2, a minimally invasive spinal fixation system 20 used to stabilize a region of the spine 2 includes a fixation rod 200 of sufficient length to extend across the vertebrae 4 to be stabilized, pedicle screws 300 for anchoring the fixation rod 200 to each corresponding vertebra 4, and a fastener 500 on each pedicle screw 300 to secure the pedicle rod 200 to the pedicle screw 300. Each pedicle screw 300 is implanted in the pedicle 6 of the corresponding vertebra 4 through a small skin incision having a length generally corresponding to a cross sectional dimension of the pedicle screw 300. In the illustrated embodiment, for example, the incision has a length of 1 cm or less, and the rod 200 is assembled with the pedicle screws 300 through a separate skin incision of 1 cm or less, as discussed further below.

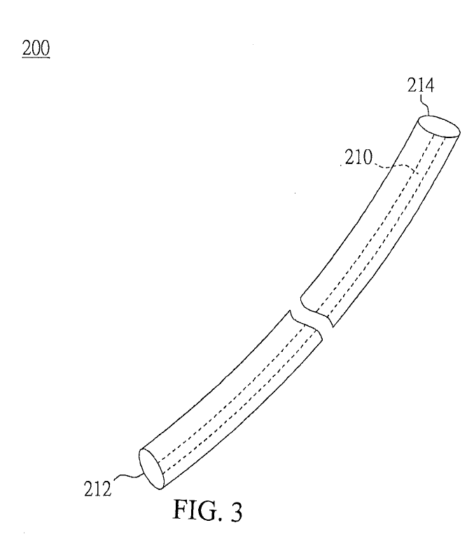

Referring to FIG. 3, the fixation rod 200 is a structure that is configured to engage a suture, where a suture is defined herein as an elongated strand or fiber such as a thread or wire. In the illustrated embodiment, the fixation rod 200 is a hollow tube, including a first end 212, a second end 214 that is opposed to the first end 212, and an interior passageway 210 that extends between the first end 212 and the second end 214. In this embodiment, the fixation rod 200 can receive a suture within the passageway 210.

The rod 200 is relatively long compared to its cross-sectional dimension. For example, in the illustrated embodiment, the rod 200 is cylindrical, and has a diameter of 5.5 mm and an axial length that corresponds to the overall length of the region of the spine 2 to be stabilized. For example, to stabilize two adjacent vertebrae 4, the rod length may be approximately 60.0 mm long. To stabilize a series of four adjacent vertebrae 4, the rod length may be approximately 150.0 mm long. The rod 200 is formed of an implantable material, and is formed of a material of sufficient strength and stiffness to provide spinal stabilization, while also being sufficiently malleable to permit shaping of the rod curvature. For example, the rod 200 may be formed of a titanium alloy such as Ti6Al4V.

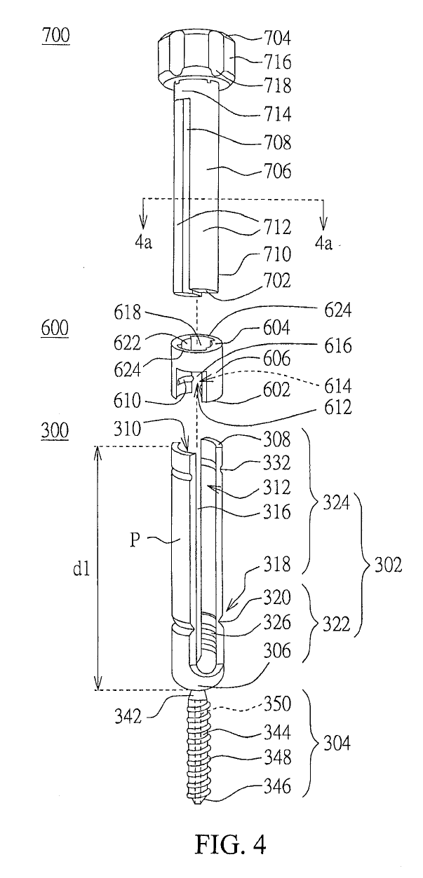

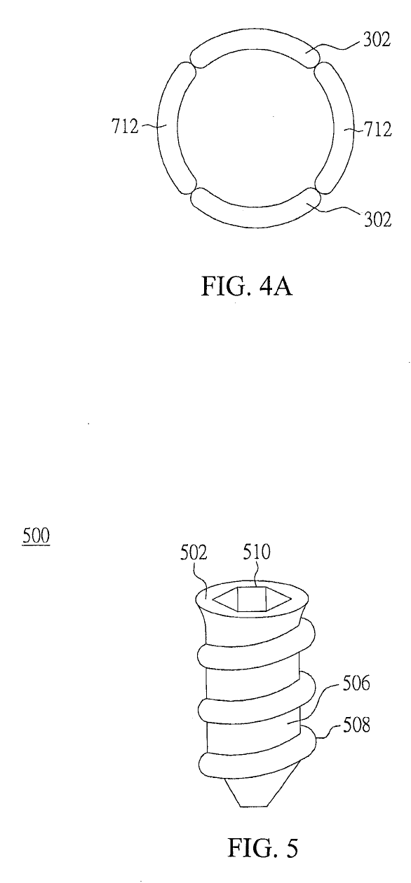

Referring to FIG. 4, a polyaxial pedicle screw 300 is used to anchor the pedicle rod 200 to each corresponding vertebra 4. Each pedicle screw 300 is dimensioned to be inserted through a skin incision of 1 cm or less and screwed into the pedicle of the corresponding vertebra 4, which lies below the skin and underlying muscle at a depth of about 5 cm for an average male. Toward this end, each pedicle screw 300 includes an elongated head 302 and a threaded tip 304.

The head 302 is generally tubular, and is formed of a single piece. The head 302 includes a closed first end 306, and an open second end 308 that is opposed to the first end 306. The head 302 is provided with a first axially-extending opening 310 that extends from the second end 308 to a location adjacent to, and spaced apart from, the first end 306. The head 302 is also provided with a second axially-extending opening 312 on an opposed side of the head 302 relative to the first opening 310. Mirroring the first opening 310, the second opening 312 extends from the second end 308 to a location adjacent to and spaced apart from the first end 306. The first and second openings 310, 312 are diametrically aligned so as to form a transverse through channel 316 through the head 302. As a result, the head 302 is generally U shaped.

The head 302 is long in an axial direction relative to its cross sectional dimension. For example, in the illustrated embodiment, the distance dl between the first end 306 and the second end 308 is in a range from 4 cm to 12 cm, whereas it has a diameter of about 1 cm. In other embodiments, the distance dl may be in a range of 5 cm to 8 cm.

The head 302 is provided with an annular breakaway region 318 that is located between the first end 306 and the second end 308. In the illustrated embodiment, the breakaway region 318 is located between the first end 306 and a midpoint P between the first and second ends 306, 308, or more specifically, at a location about midway between the point P and the first end 306. As a result, the head 302 is partitioned into two portions by the breakaway region 318. A ventral portion 322 that extends between the first end 306 and the breakaway region 318; and a dorsal portion 324 that extends between the breakaway region 318 and the second end 308. The breakaway region 318 is a region of the head 302 that is formed to be relatively structurally weak compared to the remainder of the head 302 so as to define a circumferential line along which the dorsal portion 324 can be easily separated from the ventral portion 322 upon application of sufficient force to the dorsal portion 324. In the illustrated embodiment, the breakaway region 318 is a circumferentially-extending V-shaped groove 320. In some embodiments, the spinal fixation system 20 may include a screw breaking tool 800, described further below that is configured to provide a twisting force about a longitudinal axis of the head 302 and thereby selectively separate the dorsal portion 324 from the ventral portion 322 at the groove 320. It will be appreciated that although a bending force could also be applied to the dorsal portion 324 to achieve separation, use of a twisting force will be less damaging to surrounding tissues than a bending force.

The interior surface of the ventral portion 322 is provided with threads 326 configured to engage corresponding threads 508 provided on an outer surface 506 of a fastener 500, described further below. In addition, a retention groove 332 is formed in the outer surface of the head 302 at a location adjacent the second end 308. The retention groove 332 is dimensioned and positioned so as to receive and retain a corresponding annular ridge 610 formed on an inner surface of a screw cap 600, described further below.

The threaded tip 304 of the pedicle screw 300 extends outward from the first end 306 of the head 302. More specifically, the threaded tip 304 includes a base 342 that is supported within the first end 306 of the head 302, and a shank 344 that extends from the base. The first end 306 of the head 302 is configured to permit three dimensional rotation of the threaded tip 304 relative to the head 302. The shank 344 has outer threads 348 and terminates at an apex 346. In addition, the threaded tip 304 includes an axial through hole 350 that opens at the base 342, extends through the shank 344 and opens the apex 346.

Referring to FIG. 5, the fastener 500 is a cylindrical member having external threads 508 formed on an outer surface 506. The threads 508 are configured to engage corresponding threads 326 formed on an inner surface of the ventral portion 322 of the pedicle screw head 302. In the illustrated embodiment, the fastener 500 is a set screw having a first end 502 that is configured to receive a driving tool. For example, the end 502 includes a hexagonal-shaped socket 510 suited for receiving a hex wrench, or the shaped tip 892 of an actuator tool 850 (described below). In use, the fastener 500 is secured to the ventral portion 322 of the pedicle screw head 302 so as to retain the position of the fixation rod 200 relative to the pedicle screw 300.

Referring again to FIG. 4, the spinal fixation system 20 further includes a removable screw cap 600 that is shaped and dimensioned to be secured to the pedicle screw head second end 308, to support and stabilize the head second end 308, and to serve as a guide to direct a stabilizer tool 700 (described below) during insertion of the stabilizer tool 700 into the hollow interior of the pedicle screw 300 (described below). The screw cap 600 is a hollow cylinder having an open first end 602, a closed second end 604 opposed to the first end 602, and a sidewall 606 extending between the first end 602 and the second end 604. The open first end 602 is dimensioned to receive the second end 308 of the pedicle screw head 302 therein.

The screw cap sidewall 606 is provided with a first axially-extending cap opening 612 that extends from the first end 602 to a location adjacent to, and spaced apart from, the second end 604. The screw cap sidewall 606 is also provided with a second axially-extending cap opening 614 on an opposed side of the sidewall 606 relative to the first cap opening 612. Mirroring the first cap opening 612, the second cap opening 614 extends from the first end 602 to a location adjacent to and spaced apart from the second end 604. The first and second cap openings 612, 614 are diametrically aligned so as to form a transverse through channel 616 through the screw cap 600. When the screw cap 600 is disposed on the second end 308 of the pedicle screw head 302, the screw cap transverse through channel 616 can be aligned with the pedicle screw through channel 316, whereby the axial length of the combined through channel 316, 616 is maximized.

The screw cap 600 includes an inwardly-protruding annular ridge 610 formed on an interior surface of the sidewall 606 that is sized and positioned to permit engagement with the cap retention groove 332 formed on the pedicle screw second end 308. The protruding ridge 610 extends about the inner circumference of the sidewall, and cooperates with the retention groove 332 to maintain the screw cap 600 on the pedicle screw second end 308.

In addition, the second end 604 of the screw cap 600 includes a central opening 618. The central opening 618 has an irregular shape, including a generally circular central portion 622 and an elongated portion 624 positioned along each opposed side of, and intersecting, the central portion 622. In the illustrated embodiment, the central portion 622 is shaped and dimensioned to permit passage of surgical tools through the screw cap 600 and into the interior space of the pedicle screw head 302. In addition, the elongated portions 624 are shaped and dimensioned to receive leg portions 712 of the stabilizer tool 700 when the stabilizer tool 700 is inserted into the hollow interior of the pedicle screw 300 (described below). It should be noted that the elongated portions 624 of the central opening 618 are located along a periphery of the second end 604 so as to overlie respective first and second cap openings 612, 614. This configuration ensures that the leg portions 712 of the stabilizer tool 700 are aligned with respective first and second openings 310, 312 of the pedicle screw head 302 after assembly of the pedicle screw 300, cap 600 and stabilizer tool 700, as discussed further below.

The spinal fixation system 20 further includes the stabilizer tool 700 which is a hollow cylinder including an open first end 702, a closed second end 704 opposed to the first end 702, and a sidewall 706 extending between the first end 702 and the second end 704. The sidewall 706 is formed having an outer diameter that corresponds to that of the pedicle screw head 302 and diametrically opposed openings 708, 710 that extend axially from the first end 702 to a location adjacent the second end 704. The openings 708, 710 provide the sidewall 706 with a generally U-shape, including leg portions 712 that are joined by an annular base portion 714. A grip portion 716 is disposed between the base portion 714 and the second end 704 that has a larger outer diameter than the base portion 714, and includes surface features such as axially-extending grooves 718 to improve gripability. In addition, the second end 704 includes a central opening (not shown in FIG. 4) through which tools can be inserted.

When the stabilizer tool 700 is assembled with the cap 600 and pedicle screw 300, the leg portions 712 reside within the openings 310, 312 of the pedicle screw head 302 (see FIG. 4A). The stabilizer tool 700 is used to position the pedicle rod 200 within the interior space of the pedicle screw head 302 during implantation of the spinal fixation system 20. In addition, the stabilizer tool 700 is used to maintain the position of the pedicle rod 200 while the fastener 500 is used to secure the pedicle rod 200 to the ventral portion 322 of the pedicle screw head 302, and to reinforce the dorsal portion 324 during separation of the dorsal portion 324 from the ventral portion 322 after implantation, as discussed further below.

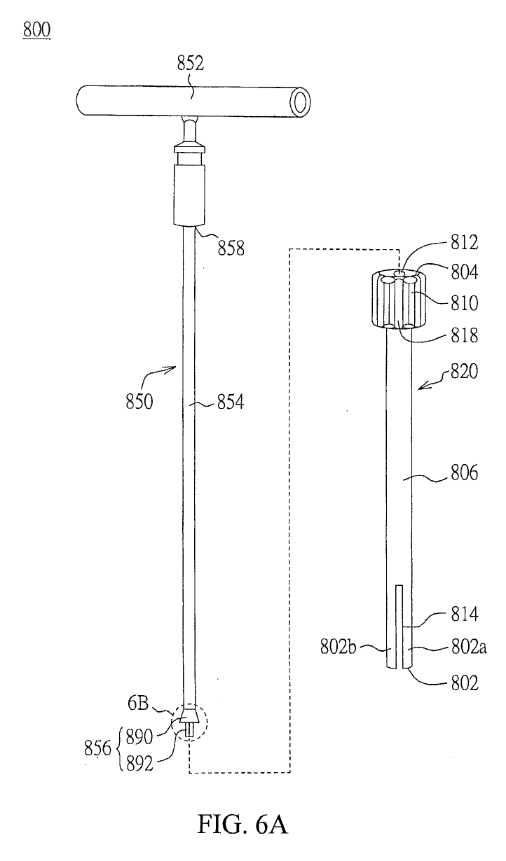

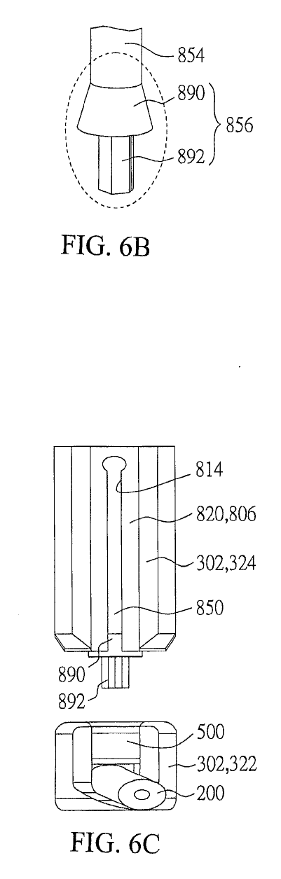

Referring to FIGS. 6a-6c, the spinal fixation system 20 further includes the screw breaking tool 800 that is configured to be received within the interior space of the pedicle screw head 302 and is used to remove the dorsal portion 324 of the pedicle screw head 302 once the pedicle screw ventral portion 322 and pedicle rod 200 are correctly positioned and mutually fixed. The screw breaking tool is 800 includes a sleeve 820 and a T-shaped actuator 850 shaped and dimensioned to be received within the sleeve 820 (FIG. 6a). The sleeve 820 is a hollow cylinder that includes an open first end 802, a second end 804 opposed to the first end 802, and a sidewall 806 extending between the first end 802 and the second end 804. A pair of slots 814 (only one slot 814 is shown) extend from the first end 802 toward a mid portion of the sleeve 820. The slots 814 divided the first end 802 into two end portions 802a, 802b. A grip region 810 is provided on the second end 804 that has a larger outer diameter than the sidewall 806, and includes surface features such as axially-extending grooves 818 to improve gripability. In addition, the second end 804 includes a central opening 812 through which tools, including the actuator 850, can be inserted. The axial length of sleeve 820 is greater than that of an assembly of the pedicle screw 300, cap 600 and stabilizer tool 700.

The actuator 850 includes a shank 854 having a first end 856 and a second end 858. A handle 852 is fixed to the second end 858, giving the actuator its T-shape. The shank first end 856 includes a flared portion 890, and a shaped portion 892 that extends coaxially from the flared portion 890 (FIG. 6b). The shaped portion 892 has an outer cross sectional dimension that is less than that of the flared portion 890 and shank 854, and includes surface features that enable it to engage the socket 510 of the fastener 500. For example, in the illustrated embodiment, the shaped portion 892 is hexagonal in cross-sectional shape so as to engage the hexagonal socket 510 of the fastener 500. The flared portion 890 has an outer dimension that is greater than that of the sleeve sidewall 806 and the diameter of the interior space of the pedicle screw head 302. When the actuator 850 is assembled within the sleeve 820 with the flared portion 890 protruding beyond the sleeve first end 802, the sleeve 820 can be inserted into the screw head 302, for example to secure the fastener 500 to the screw head 302. By drawing the actuator 850 upward so that at least a portion of the flared portion 890 is disposed within first end of the sleeve 820, the flared portion 890 causes the two end portions 802a, 802b to slightly separate. By this action, the outer wall of the sleeve 820 is compressed against the inner wall of the pedicle screw head dorsal portion 324. Due to frictional engagement of the sleeve 820 with the pedicle screw head 302, by rotating the actuator 850 about its longitudinal axis, a twisting force is applied to the dorsal portion 324 of the screw head 302. Upon application of sufficient force, the dorsal portion 324 of the screw head 302 can be separated from the ventral portion 322 along the breakaway line 318 (FIG. 6c).

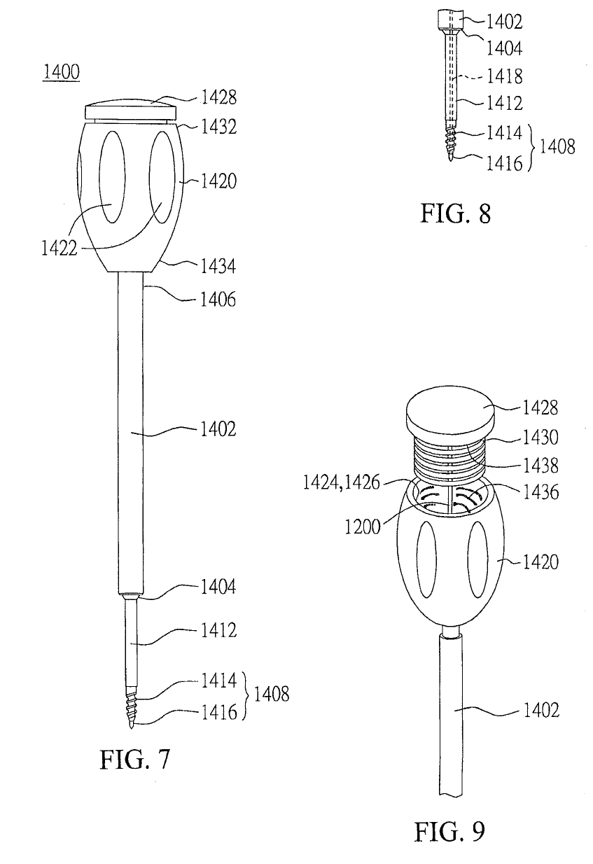

Referring to FIGS. 7-9, an awl 1400 is used to prepare each vertebra 4 for implantation, as described further below. The awl 1400 includes an elongated cylindrical shaft 1402 having a first end 1404, and a second end 1406 opposed to the first end 1404.

The awl 1400 is formed having an extension 1412 that extends from the first end 1404 of the shaft. The extension 1412 is elongated to facilitate stabilization of the direction of implantation, and terminates in a cutting tip 1408 that has two cutting portions: A drill portion 1416 on a leading end (terminus) thereof; and a tap portion 1414 disposed between the drill portion 1416 and the shaft 1402. The drill portion 1416 includes cutting surfaces configured to form a hole in bone. The tap portion 1414 includes tap threads configured to form an internal screw thread in the bone along the surface of the hole formed by the drill portion 1416. The tap portion 1414 and drill portion 1416 are slightly axially spaced apart. In the illustrated embodiment, the extension 1412, drill portion 1416, and tap portion 1414 are all formed as one piece with, or fixed to, the shaft 1402. However, in some embodiments, one or more of the extension 1412, the drill portion 1416, and/or the tap portion 1414 are detachable.

A handle 1420 is fixed to the second end 1406 of the shaft 1402. The handle 1420 is generally ovoid in shape, and is provided with surface features such as circumferentially spaced grooves 1422 that improve handle gripability. The handle 1420 is hollow and includes a distal end 1434 that is fixed to the shaft first end 1406, and a proximal end 1432 that is opposed to the distal end 1434. The proximal end 1432 is open in order to provide access to the interior space 1436 of the handle 1420. At least a portion of the interior surface 1424 of the handle 1420 is formed having screw threads 1426. In addition, the handle 1420 includes a detachable cap 1428 that closes the open proximal end 1432. The cap 1428 has exterior threads 1430 that are configured to engage the handle interior screw threads 1426, whereby the cap 1428 can be selectively retained on the proximal end 1432. An inward-facing surface 1438 of the cap 1428 is configured to releasably engage an end of a Kirshner pin 1200. For example, in some embodiments, the cap inward-facing surface 1438 may be formed having an opening (not shown) dimensioned to receive an end of a Kirshner pin 1200 in a press fit engagement.

In addition, the awl 1400 includes an axially extending through hole 1418 that extends from the open interior space 1436 of the handle 1420 to the cutting tip 1408, and is dimensioned to receive a Kirshner pin 1200.

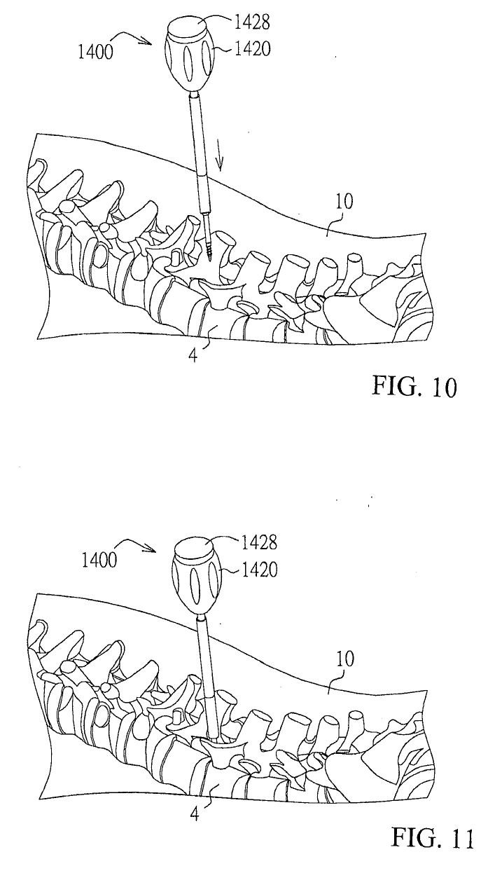

Referring to FIGS. 10-22, an example of a method of implanting a pedicle screw 300 into a vertebra 4 using the awl 1400 will now be described.

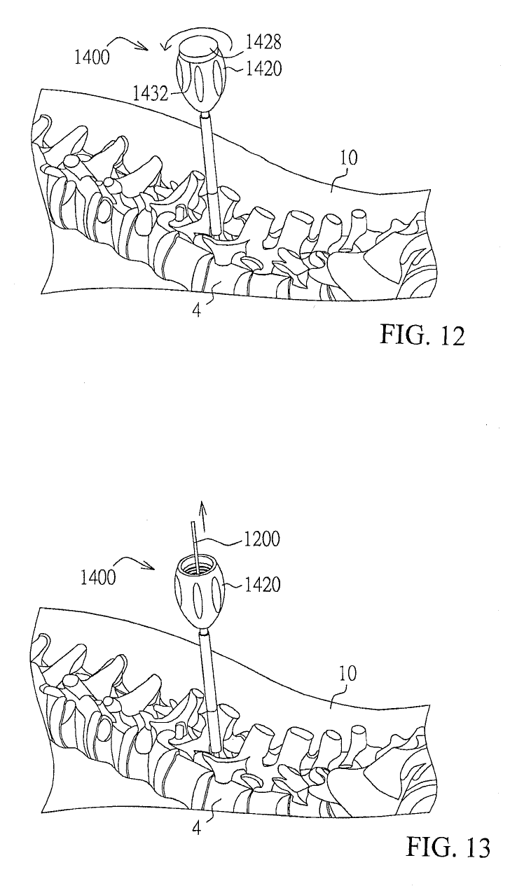

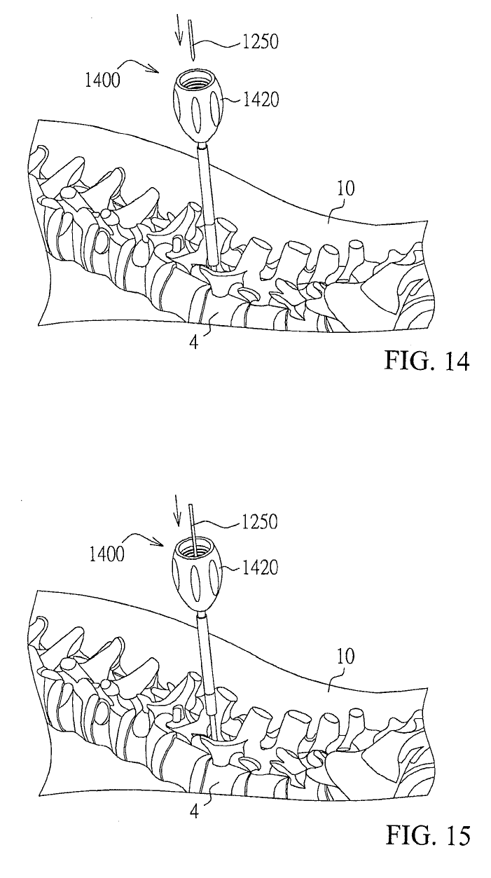

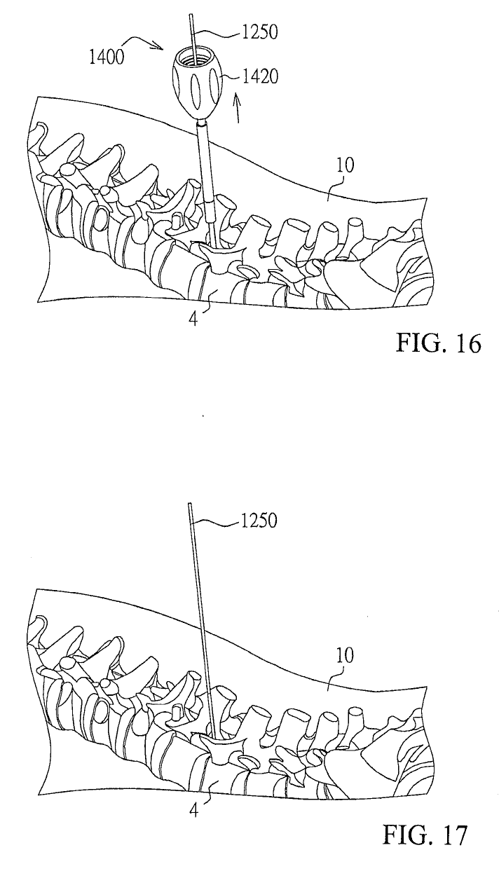

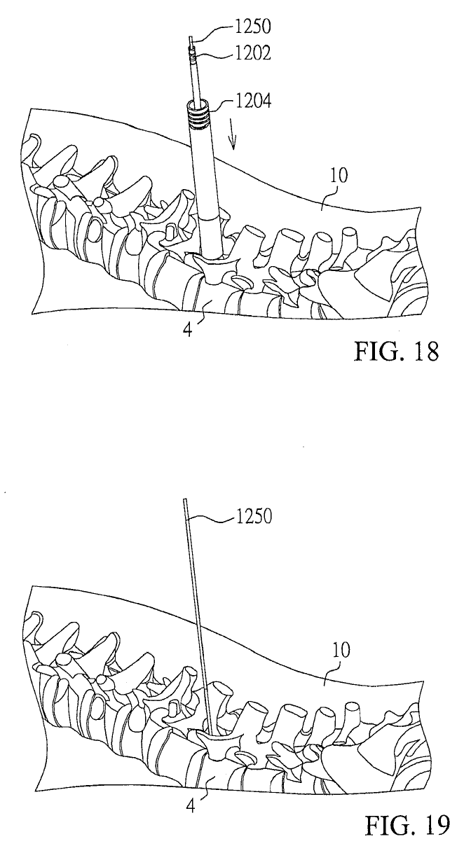

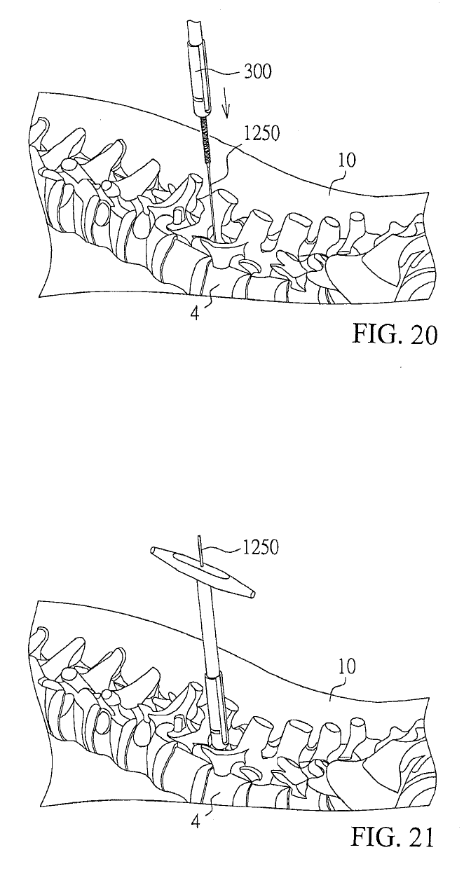

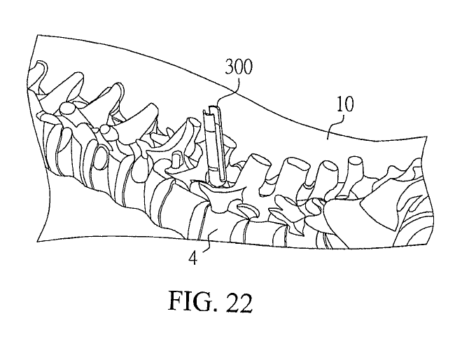

Step 1. Provide an incision through the skin 10 overlying the vertebra 4. In general, the incision length corresponds to the outer diameter of the pedicle screw 300, and may be slightly less due to the pliability of skin. In the illustrated embodiment, the pedicle screw 300 is approximately 1 cm in diameter, whereby an incision of at most 1 cm is required to accommodate pedicle screw 300.
Step 2. Referring to FIG. 10, prepare the vertebra 4 to receive the pedicle screw 300 by forming a threaded hole in the pedicle 6. The awl 1400, including a first Kirshner pin 1200 disposed within the awl's through hole 1418, is inserted into the incision and is used to locate the pedicle 6 and form the threaded hole therein. Then, a hammer (not shown) is used to break the cortical bone in order to make an entry hole. Specifically, the awl 1400 is inserted subcutaneously into the vertebra 4 until it touches the pedicle bone 6. Correct positioning is verified using an imaging device such as a C-arm or fluoroscope.
Step 3. Referring to FIG. 11, after correct positioning of the awl 1400 is confirmed, rotate the awl so that the cutting tip 1408 is screwed into the vertebra 4. In particular, the awl is rotated so that the drill portion 1416 forms a hole in the vertebra, and then further rotated so that the tap portion 1414 forms an internal screw thread in the hole formed by the drill portion 1416. Again, positioning and orientation are checked with an imaging device.
Step 4. Referring to FIG. 12, remove the cap 1428 from the proximal end 1432 of the awl handle 1420.
Step 5. Referring to FIG. 13, remove the first Kirshner pin 1200 from the awl 1400.
Step 6. Referring to FIG. 14, insert a second Kirshner pin 1250 through the axial passageway 1418 of the awl and into the vertebra 4 at the desired implantation location. The second Kirshner pin 1250 is longer than the first Kirshner pin 1200. In some embodiments, the second Kirshner pin 1250 is about 30 cm in length.
Step 7. Referring to FIG. 15, urge the second Kirshner 1250 pin deeper into the vertebra 4, and confirm its stability.
Step 8. Referring to FIGS. 16 and 17, remove the awl 1400 from the incision, leaving the second Kirshner pin 1250 in place.
Step 9. Referring to FIG. 18, insert a series of dilation cannulas 1202, 1204 into the incision over the second Kirshner pin 1250, starting with a relatively small-diameter cannula 1202, and each successive cannula having a slightly larger outer diameter. Although only two cannulas 1202, 1204 are shown, six to eight cannulas may be used in order to dilate the skin 10, muscle and other soft tissues in the vicinity of the second Kirshner pin 1250 and create space for insertion of the pedicle screw 300 into the body. The second Kirshner pin 1250 is used to stabilize and direct each respective dilation cannula 1202, 1204 during dilation.
Step 10. Referring to FIG. 19, after dilation is completed, withdraw the dilation cannulas 1202, 1204, leaving the second Kirshner pin 1250 in place.
Step 11. Referring to FIGS. 20-21, implant the pedicle screw 300 in the pre-threaded drill hole of the vertebra 4 by passing it along the second Kirshner pin 1250. Specifically, the pedicle screw 300 is loaded onto the second Kirshner pin 1250 so that the second Kirshner pin 1250 is received within the shank axial through hole 350 and the interior space of the pedicle screw head 302. The second Kirshner pin 1250 serves to stabilize and direct the pedicle screw shank 344 so that the threads 348 on the shank 344 engage with and are screwed onto the drill hole threads. In the illustrated embodiment, a driving tool is used to rotate the pedicle screw 300, screwing the pedicle screw into the hole in the vertebra 4.
Step 12. Referring to FIG. 22, an implanted pedicle screw is illustrated.

A minimally invasive method for achieving spinal stabilization using spinal fixation system 20 is described in co-pending US application Ser. No. ______ and is incorporated by reference herein.

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art. Moreover, while a working example of the present invention has been described above, the present invention is not limited to the working example described above, but various design alterations may be carried out without departing from the present invention as set forth in the claims.

What is claimed is:

1. A bone awl, comprising
a shaft having a first end and a second end opposed to the first end,
a handle fixed to the second end, and
an extension extending from the first end that includes a first cutting portion configured to form a hole in bone and a second cutting portion configured to form an internal screw thread in the bone along the surface of the hole.

2. The bone awl of claim 1, further comprising an axial through passage that extends from the handle to the first cutting portion.

3. The bone awl of claim 1 wherein the first cutting portion defines a terminus of the awl, and the second cutting portion is disposed between the first cutting portion and the shaft first end.

4. The bone awl of claim 1 wherein the first and second cutting portions are selectively detachable from the shaft.

5. The bone awl of claim 1 wherein the second cutting portion includes tap threads.

6. The bone awl of claim 1 wherein an external surface of the handle includes surface features configured to improve gripability.

7. The bone awl of claim 1, wherein the handle includes a detachable cap.

8. The bone awl of claim 7 wherein the detachable cap is configured to releasably engage an end of a Kirshner pin.

9. The bone awl of claim 1, wherein the handle is hollow and includes
a distal end that is fixed to the shaft second end,
a proximal end that is opposed to the distal end, the proximal end being open to provide access to the interior space of the handle, and
a detachable cap that closes the open proximal end.

10. The bone awl of claim 9 wherein the interior space is threaded, and the cap includes threads configured to engage the threads of the interior space.

11. The bone awl of claim 9 wherein the cap is configured to releasably engage an end of a Kirshner pin.

12. The bone awl of claim 2, further including a Kirshner pin disposed within the axial through passage.

13. A method of implanting a pedicle screw into a vertebra using a surgical awl comprises the following method steps:
providing the pedicle screw;
providing the surgical awl, the surgical awl including
a hollow body including a proximal end, and a distal end opposed to the proximal end, the proximal end including a handle, and the distal end including a first cutting portion configured to form a hole in bone and a second cutting portion configured to form an internal screw thread in the bone along the surface of the hole, and
a detachable cap formed on the handle,
a first Kirshner pin extending through the hollow body to the distal end;
forming an incision through the skin overlying the vertebra;
inserting the awl into the incision until the distal end contacts the vertebra;
rotate the awl so that the first cutting portion forms a hole in the vertebra, and the second cutting portion forms an internal screw thread in the hole formed by the first cutting portion;
removing the cap from a proximal end of the awl;
removing the first Kirshner pin from the awl;
inserting a second Kirshner pin through the awl and into the vertebra at the desired implantation location;
removing the awl from the incision, leaving the second Kirshner pin in place; and
implanting the pedicle screw in the pre-threaded drill hole of the vertebra by passing it along the second Kirshner pin and screwing the pedicle screw into the hole in the vertebra.

14. The method of claim 13, wherein the cap is configured to detachably retain an end of the first Kirshner pin, and when the cap is removed from the proximal end of the awl, the first Kirshner pin is removed along with the cap.

15. The method of claim 13, further comprising a tissue dilation step following the removal of the awl from the incision, the tissue dilation step comprising
inserting a series of dilation cannulas into the incision over the second Kirshner pin, starting with a relatively small-diameter cannula, and each successive cannula having a slightly larger outer diameter;
after dilation is completed, withdrawing the dilation cannulas and leaving the second Kirshner pin in place.

16. The method of claim 13, further comprising the step of verifying correct awl positioning using an imaging device following the step of inserting the awl into the incision.

17. A method of using a surgical awl to form a tapped hole in bone comprises the following method steps:
providing the surgical awl, the surgical awl including
a proximal end, and
a distal end opposed to the proximal end, the distal end including a first cutting portion configured to form a hole in bone and a second cutting portion configured to form an internal screw thread in the bone along the surface of the hole;
placing the distal end against a surface of the bone;
rotating the awl so that the first cutting portion forms a hole in the bone; and
after forming the hole in the bone, further rotating the awl so that the second cutting portion forms an internal screw thread in the hole formed by the first cutting portion.
